SEQUENCE LISTING (18 sequences)

SEQ ID NO: 1 - DNA, 32 bp, Artificial Sequence
  oligonucleotide
ccgtggtccagtttatcgttattaccaaaggt

SEQ ID NO: 2 - DNA, 32 bp, Artificial Sequence
  oligonucleotide
ccctttccagtacgcttcgccgttcgcgcgcg

SEQ ID NO: 3 - DNA, 50 bp, Artificial Sequence
  oligonucleotide
  misc_feature 35: n=tetrahydrofuran
attttctctggatggtatgcccggtaaacagatgngtattgatgccgatt

SEQ ID NO: 4 - DNA, 33 bp, Artificial Sequence
  oligonucleotide
caaaacgtgttaaagatggtgatgtgattgccg

SEQ ID NO: 5 - DNA, 33 bp, Artificial Sequence
  oligonucleotide
aaggagagaccactctgctttttgtttggcata

SEQ ID NO: 6 - DNA, 54 bp, Artificial Sequence
  oligonucleotide
  misc_feature 37: n=tetrahydrofuran
caaaacgtgttaaagatggtgatgtgattgccgttantggtatcactggtgaag

SEQ ID NO: 7 - DNA, 35 bp, Artificial Sequence
  oligonucleotide
ctttagttgtagtttcaagtctaagtagctcagca

SEQ ID NO: 8 - DNA, 35 bp, Artificial Sequence
  oligonucleotide
cattaatttaaccgtatcaccatcaatcgctttaa

SEQ ID NO: 9 - DNA, 45 bp, Artificial Sequence
  oligonucleotide
  misc_feature 30: n=tetrahydrofuran
agtttcaagtctaagtagctcagcaaatgnatcacaaacagataa

SEQ ID NO: 10 - DNA, 35 bp, Artificial Sequence
  oligonucleotide
cgtcttacaacgcagtaactacgcactatcattca

SEQ ID NO: 11 - DNA, 35 bp, Artificial Sequence
  oligonucleotide
caaaatgacattcccacatcaaatgatgcgggttg

SEQ ID NO: 12 - DNA, 35 bp, Artificial Sequence
  oligonucleotide
ctgcggaggctaactatgtcaaaaatcatgaacct

SEQ ID NO: 13 - DNA, 35 bp, Artificial Sequence
  oligonucleotide
acattcaaaatccctttatgaagcggctgaaaaaa

SEQ ID NO: 14 - DNA, 35 bp, Artificial Sequence
  oligonucleotide
atgtaattcctccacatctcattaaatttttaaat

SEQ ID NO: 15 - DNA, 46 bp, Artificial Sequence
  oligonucleotide
  misc_feature 31: n=tetrahydrofuran
tgacattcccacatcaaatgatgcgggttgngttaattgarcaagt

SEQ ID NO: 16 - DNA, 33 bp, Artificial Sequence
  oligonucleotide
gcaaaagatagttcaactaatcaatatgtaagt

SEQ ID NO: 17 - DNA, 33 bp, Artificial Sequence
  oligonucleotide
acttcatctgggctagctaaaatttcacgggca

SEQ ID NO: 18 - DNA, 52 bp, Artificial Sequence
  oligonucleotide
  misc_feature 36: n=tetrahydrofuran
tcatctgggctagctaaaatttcacgggcactttgnctaagatctgctttta

(19) United States
(12) Patent Application Publication
(10) Pub. No.: US 2017/0335379 A1
(43) Pub. Date: Nov. 23, 2017

(54) DETECTION OF NUCLEIC ACIDS IN CRUDE MATRICES

(71) Applicant: Alere San Diego Inc., San Diego, CA (US)

(72) Inventor: Niall A. Armes, Helions Bumpstead (GB)

(21) Appl. No.: 15/612,418

(22) Filed: Jun. 2, 2017

Related U.S. Application Data
(63) Continuation of application No. 13/498,035, filed on Nov. 15, 2012, now abandoned.
(60) Provisional application No. 61/245,758, filed on Sep. 25, 2009.

(51) Int. Cl. C12Q 1/68

(52) U.S. Cl. CPC C12Q 1/6846

(57) ABSTRACT

A method includes contacting a crude matrix with components of an isothermal nucleic acid amplification reaction for a target nucleic acid species, thereby providing a mixture; incubating the mixture under conditions sufficient for the isothermal nucleic acid amplification reaction to proceed, thereby providing a product; and determining whether an indicator of the target nucleic acid species is present in the product.

### CLAIM OF PRIORITY

This application claims priority to U.S. patent application Ser. No. 61/245,758, filed on Sep. 25, 2009, the entire contents of which are incorporated herein by reference.

### TECHNICAL FIELD

This disclosure relates to detection of nucleic acids by amplification methods in crude matrices.

### BACKGROUND

Isothermal amplification methods are able to amplify nucleic acid targets in a specific manner from trace levels to very high and detectable levels within a matter of minutes. Such isothermal methods, e.g., Recombinase Polymerase Amplification (RPA), can broaden the application of nucleic acid based diagnostics into emerging areas such as point-of-care testing, and field and consumer testing. The isothermal and broad temperature range of the technologies can allow users to avoid the use of complex power-demanding instrumentation.

### SUMMARY

The present disclosure is based, at least in part, on the discovery that various pathogenic organisms can be detected in crude matrices without nucleic acid extraction and/or purification. The use of crude matrices without nucleic acid extraction and/or purification can add the advantage of simple sample preparation to the advantages of isothermal nucleic acid amplification methods as described above. In some cases, simple treatment such as alkaline lysis or lytic enzyme treatment is sufficient for detection. In some other cases, target nucleic acid sequences of the organisms could be detected at high sensitivity without any need to pre-treat the sample with conventional lysis solutions. Instead, contacting the sample with an isothermal amplification reaction is sufficient to detect the organisms at high sensitivity.

In one aspect, the disclosure features a method that includes contacting a crude matrix with components of an isothermal nucleic acid amplification reaction for a target nucleic acid species, thereby providing a mixture; incubating the mixture under conditions sufficient for the isothermal nucleic acid amplification reaction to proceed, thereby providing a product; and determining whether an indicator of the target nucleic acid species is present in the product.

In another aspect, the disclosure features a method that includes contacting a crude matrix with components of a nucleic acid amplification reaction for a target nucleic acid species, thereby providing a mixture; maintaining the mixture at a temperature of less than 95° C. (e.g., less than 90° C., less than 85° C., less than 80° C., less than 75° C., less than 70° C., less than 65° C., less than 60° C., less than 55° C., less than 50° C., less than 45° C., or less than 40° C.) for a time sufficient to allow the nucleic acid amplification reaction to proceed, thereby providing a product; and determining whether an indicator of the target nucleic acid species is present in the product.

In another aspect, the disclosure features a method that includes contacting a crude matrix with components of a nucleic acid amplification reaction for a target nucleic acid species, thereby providing a mixture; varying a Celsius-scale temperature of the mixture by less than 30% (e.g., less than 25%, less than 20%, less than 15%, less than 10%, or less than 5%) or by less than 20° C. (e.g., less than 15° C., less than 10° C., less than 5° C., less than 2° C., or less than 1° C.) for a time sufficient to allow the nucleic acid amplification reaction to proceed, thereby providing a product; and determining whether an indicator of the target nucleic acid species is present in the product.

In another aspect, the disclosure features a method that includes performing an isothermal reaction of a mixture to provide a product, the mixture comprising a crude matrix and components of a nucleic acid amplification reaction for a target nucleic acid species; and determining whether an indicator of the target nucleic acid species is present in the product.

In another aspect, the disclosure features a method, that includes reacting a mixture at a temperature of at most 80° C. (e.g., at most 75° C., at most 70° C., at most 65° C., at most 60° C., at most 55° C., at most 50° C., at most 45° C., or at most 40° C.) to provide a product, the mixture comprising a crude matrix and components of a nucleic acid amplification reaction for a target nucleic acid species; and determining whether an indicator of the target nucleic acid species is present in the product.

In another aspect, the disclosure features a method that includes reacting a mixture while varying a Celsius-scale temperature of the mixture by at most 30% (e.g., at most 25%, at most 20%, at most 15%, at most 10%, or at most 5%) or at most 20° C. (e.g., at most 15° C., at most 10° C., at most 5° C., at most 2° C., or at most 1° C.) to provide a product, the mixture comprising a crude matrix and components of a nucleic acid amplification reaction for a target nucleic acid species; and determining whether an indicator of the target nucleic acid species is present in the product.

In some embodiments of the above aspects, the crude matrix includes a biological sample, e.g., at least one of blood, urine, saliva, sputum, lymph, plasma, ejaculate, lung aspirate, and cerebrospinal fluid. In some embodiments, the biological sample includes at least one sample selected from a throat swab, nasal swab, vaginal swab, or rectal swab. In some embodiments, the biological sample comprises a biopsy sample.

In some embodiments of the above aspects, the crude matrix is not subjected to a lysis treatment.

In some embodiments of the above aspects, the crude matrix is not treated with a chaotropic agent, a detergent, or a lytic enzyme preparation.

In some embodiments of the above aspects, the crude matrix is not subjected to a high temperature (e.g., 80° C. or higher, 85° C. or higher, 90° C. or higher, or 95° C. or higher) thennal treatment step.

In some embodiments of the above aspects, the crude matrix is not subjected to a lysis treatment and the target nucleic acid species is aStaphylococcus (e.g., S. aureus or methicillin resistant S. aureus (MRSA)) nucleic acid.

In some embodiments of the above aspects, the crude matrix is not subjected to a lysis treatment and the target nucleic acid species is a mycoplasma nucleic acid.

In some embodiments of the above aspects, the crude matrix can be subjected to a lysis treatment. For example, treating the crude matrix with a detergent and/or a lytic enzyme such as a bacteriophage lysin (e.g., streptococcal C₁ bacteriophage lysin (PlyC)).

In some embodiments of the above aspects, the crude matrix is subjected to a lysis treatment and the target nucleic acid species is a Streptococcus (e.g., Group A Streptococcus or Group B Streptococcus) nucleic acid.

In some embodiments of the above aspects, the crude matrix is subjected to a lysis treatment and the target nucleic acid species is a Salmonella (e.g., S. typhimurium) nucleic acid.

In some embodiments of the above aspects, the target nucleic acid is a bacterial nucleic acid, e.g., from a bacterium selected from Chlamydia trachomatis, Neisseria gonorrhea, Group A Streptococcus, Group B Streptococcus, Clostridium difficile, Escherichia coli, Mycobacterium tuberculosis, Helicobacter pylori, Gardnerella vaginalis, Mycoplasma hominis, Mobiluncus spp., Prevotella spp., and Porphyromonas spp, or from another bacterium described herein.

In some embodiments of the above aspects, the target nucleic acid is a mammalian nucleic acid, e.g., a nucleic acid is associated with tumor cells.

In some embodiments of the above aspects, the target nucleic acid is a viral nucleic acid, e.g., from HIV, influenza virus, or dengue virus, or from another virus described herein.

In some embodiments of the above aspects, the target nucleic acid is a fungal nucleic acid, e.g., from Candida albicans or another fungus described herein. In some embodiments of the above aspects, the target nucleic acid is a protozoan nucleic acid, e.g., from Trichomonas or another protozoan described herein.

In some embodiments of the above aspects, the isothermal nucleic acid amplification reaction is recombinase polymerase amplification. In some embodiments, the isothermal nucleic acid amplification reaction is transcription mediated amplification, nucleic acid sequence-based amplification, signal mediated amplification of RNA, strand displacement amplification, rolling circle amplification, loop-mediated isothermal amplification of DNA, isothermal multiple displacement amplification, helicase-dependent amplification, single primer isothermal amplification, circular helicase-dependent amplification, or nicking and extension amplification reaction.

In some embodiments of the above aspects, the reaction conditions comprise polyethylene glycol (PEG), e.g., at a concentration of greater than 1%.

In another aspect, the disclosure features a method for detection of a specific DNA or RNA species in which a sample is contacted to a reaction rehydration buffer or to a hydrated reaction system without prior lysis treatment with a chaotropic agent, a detergent, without a high temperature thermal treatment step, or a lytic enzyme preparation, and is amplified to a detectable level. In some embodiments, the target nucleic acid species comprises genomic DNA of Staphylococcus aureus or MRSA. In some embodiments, the method of amplification is the Recombinase Polymerase Amplification (RPA) method. In some embodiments, polyethylene glycol is included in the rehydration buffer or fully rehydrated amplification environment at a concentration greater than 1%.

In another aspect, the disclosure features kits that include components of an isothermal nucleic acid amplification reaction; and a lytic enzyme. The components of an isothermal nucleic acid amplification reaction can include, e.g., a recombinase. In some embodiments, the lytic enzyme includes a bacteriophage lysin, e.g., streptococcal C₁ bacteriophage lysin (PlyC).

In another aspect, the disclosure features kits that include components of an isothermal nucleic acid amplification reaction; and a lateral flow or microfluidic device (e.g. for detection of a reaction product). The components of an isothermal nucleic acid amplification reaction can include, e.g., a recombinase.

In another aspect, the disclosure features kits that include components of an isothermal nucleic acid amplification reaction; and a swab (e.g., for obtaining a biological sample). The components of an isothermal nucleic acid amplification reaction can include, e.g., a recombinase.

In some embodiments of any of the above kits, the kit does not include reagents for nucleic acid purification or extraction, e.g., a chaotropic agent and/or a nucleic acid-binding medium.

As used herein, a “crude matrix” is a matrix that includes nucleic acids from a biological source, wherein the matrix has not been subjected to nucleic acid extraction and/or purification. In some embodiments, the biological source includes cells and/or a biological sample (e.g., from a patient) and/or an environmental sample. The cells and/or biological sample and/or environmental sample can be unlysed or subjected to a lysis step.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS FIGS. 1A-B are line graphs depicting detection of S. typhimurium at 10,000, 1000, and 100 cfu without lysis (1A) or following alkaline lysis (1B).

FIG. 2 is a line graph depicting detection of Strep A without lysis (NO LYSIS), treated with mutanolysin and lysozyme (ML/LZ), treated with PlyC (PLYC), or treated with mutanolysin, lysozyme, and PlyC (ML/LZ/PLYC).

FIG. 3 is a line graph depicting detection of S. aureus in patient samples treated with 0, 1, 2, or 3 units of lysostaphin.

FIG. 4 is a line graph depicting detection of S. aureus in patient samples boiled for 45 minutes (Boil), treated with lysostaphin and boiled for 5 minutes (Lysostaphin), or incubated in water at room temperature for 45 minutes. Samples were compared to positive control with 50 or 1000 copies of the target nucleic acid.

FIG. 5 is a line graph depicting detection of S. aureus in patient samples that were unlysed (Unlysed) or lysed with lysotaphin and extracted (Cleaned). Samples were compared to positive control with 50 or 1000 copies of the target nucleic acid.

FIG. 6 is a line graph depicting detection of unlysed methicillin-resistant Staphylococcus aureus (MRSA) samples with ˜10 (10 bacteria) or ˜100 (100 bacteria) organisms. Samples were compared to positive control with 50 copies of the target nucleic acid (50 copies PCT product) or water as a negative control (NTC).

FIG. 7 is a line graph depicting detection of unlysed mycoplasma at 50, 100, or 1000 cfu or a medium control.

### DETAILED DESCRIPTION

The present disclosure provides methods for isothermal amplification of nucleic acids in crude matrices for detection of nucleic acid targets.

In some embodiments, a crude matrix is contacted with components of an isothermal nucleic acid amplification reaction (e.g., RPA) for a target nucleic acid species to provide a mixture. The mixture is then incubated under conditions sufficient for the amplification reaction to proceed and produce a product that is evaluated to determine whether an indicator of the target nucleic acid species is present. If an indicator of the target nucleic acid species is found in the product, one can infer that the target nucleic acid species was present in the original crude matrix.

In some embodiments, the crude matrix includes a biological sample, e.g., a sample obtained from a plant or animal subject. As used herein, biological samples include all clinical samples useful for detection of nucleic acids in subjects, including, but not limited to, cells, tissues (for example, lung, liver and kidney), bone marrow aspirates, bodily fluids (for example, blood, derivatives and fractions of blood (such as serum or buffy coat), urine, lymph, tears, prostate fluid, cerebrospinal fluid, tracheal aspirates, sputum, pus, nasopharyngeal aspirates, oropharyngeal aspirates, saliva), eye swabs, cervical swabs, vaginal swabs, rectal swabs, stool, and stool suspensions. Other suitable samples include samples obtained from middle ear fluids, bronchoalveolar lavage, tracheal aspirates, sputum, nasopharyngeal aspirates, oropharyngeal aspirates, or saliva. In particular embodiments, the biological sample is obtained from an animal subject. Standard techniques for acquisition of such samples are available. See for example, Schluger et al., J. Exp. Med. 176:1327-33 (1992); Bigby et al., Am. Rev. Respir. Dis. 133:515-18 (1986); Kovacs et al., NEJM 318:589-93 (1988); and Ognibene et al., Am. Rev. Respir. Dis. 129:929-32 (1984).

In some embodiments, the crude matrix includes an environmental sample, e.g., a surface sample (e.g., obtained by swabbing or vacuuming), an air sample, or a water sample.

In some embodiments, the crude matrix includes isolated cells, e.g., animal, bacterial, fungal (e.g., yeast), or plant cells, and/or viruses. The isolated cells can be cultured using conventional methods and conditions appropriate for the type of cell cultured.

The crude matrix can be contacted with the nucleic acid amplification components essentially as-is or subjected to one or more pre-treatment steps that do not include nucleic acid extraction and/or purification. In some embodiments, the crude matrix is subjected to lysis, e.g., with a detergent and/or a lytic enzyme preparation. In some embodiments, the crude matrix is not subjected to treatment with a chaotropic agent, a detergent, or a lytic enzyme preparation, and the crude matrix is not subjected to a high-temperature (e.g., greater than 80° C., greater than 85° C., greater than 90° C., or greater than 95° C.). Under any or all of the above conditions, a target nucleic acid present in the crude matrix is accessible to the isothermal nucleic acid amplification machinery such that amplification can occur.

Numerous nucleic acid amplification techniques are known, including recombinase polymerase amplification (RPA), transcription mediated amplification, nucleic acid sequence-based amplification, signal mediated amplification of RNA technology, strand displacement amplification, rolling circle amplification, loop-mediated isothermal amplification of DNA, isothermal multiple displacement amplification, helicase-dependent amplification, single primer isothermal amplification, circular helicase-dependent amplification, and nicking and extension amplification reaction (see US 2009/0017453) for example. Polymerase chain reaction is the most widely known method but differs in that it requires use of thermal cycling to cause separation of nucleic acid strands. These and other amplification methods are discussed in, for example, VanNess et al., PNAS 2003. vol 100, no 8, p 4504-4509; Tan et al., Anal. Chem. 2005, 77, 7984-7992; Lizard ct al., Nature Biotech. 1998, 6, 1197-1202; Notomi ct al., NAR 2000, 28, 12, e63; and Kurn et al., Clin. Chem. 2005, 51:10, 1973-1981. Other references for these general amplification techniques include, for example, U.S. Pat. Nos. 7,112,423; 5,455,166; 5,712,124; 5,744,311; 5,916,779; 5,556,751; 5,733,733; 5,834,202; 5,354,668; 5,591,609; 5,614,389; 5,942,391; and U.S. patent publications numbers US20030082590; US20030138800; US20040058378; and US20060154286. All of the above documents are incorporated herein by reference.

RPA is one exemplary method for isothermal amplification of nucleic acids. RPA employs enzymes, known as recombinases, that are capable of pairing oligonucleotide primers with homologous sequence in duplex DNA. In this way, DNA synthesis is directed to defined points in a sample DNA. Using two gene-specific primers, an exponential amplification reaction is initiated if the target sequence is present. The reaction progresses rapidly and results in specific amplification from just a few target copies to detectable levels within as little as 20-40 minutes. RPA methods are disclosed, e.g., in U.S. Pat. No. 7,270,981; U.S. Pat. No. 7,399,590; U.S. Pat. No. 7,777,958; U.S. Pat. No. 7,435,561; US 2009/0029421; and PCT/US2010/037611, all of which are incorporated herein by reference.

RPA reactions contain a blend of proteins and other factors that are required to support both the activity of the recombination element of the system, as well as those which support DNA synthesis from the 3′ ends of oligonucleotides paired to complementary substrates. The key protein component of the recombination system is the recombinase itself, which may originate from prokaryotic, viral or eukaryotic origin. Additionally, however, there is a requirement for single-stranded DNA binding proteins to stabilize nucleic acids during the various exchange transactions that are ongoing in the reaction. A polymerase with strand-displacing character is required specifically as many substrates are still partially duplex in character. In some embodiments where the reaction is capable of amplifying from trace levels of nucleic acids, in vitro conditions that include the use of crowding agents (e.g., polyethylene glycol) and loading proteins can be used. An exemplary system comprising bacteriophage T4 UvsX recombinase, bacteriophage T4 UvsY loading agent, bacteriophage T4 gp32 and Bacillus subtilis polymerase I large fragment has been reported.

The components of an isothermal amplification reaction can be provided in a solution and/or in dried (e.g., lyophilized) form. When one or more of the components are provided in dried form, a resuspension or reconstitution buffer can be also be used.

Based on the particular type of amplification reaction, the reaction mixture can contain buffers, salts, nucleotides, and other components as necessary for the reaction to proceed. The reaction mixture can be incubated at a specific temperature appropriate to the reaction. In some embodiments, the temperature is maintained at or below 80° C., e.g., at or below 70° C., at or below 60° C., at or below 50° C., at or below 40° C., at or below 37° C., or at or below 30° C. In some embodiments, the reaction mixture is maintained at room temperature. In some embodiments, the Celsius-scale temperature of the mixture is varied by less than 25% (e.g., less than 20%, less than 15%, less than 10%, or less than 5%) throughout the reaction time and/or the temperature of the mixture is varied by less than 15° C. (e.g., less than 10° C., less than 5° C., less than 2° C., or less than 1° C.) throughout the reaction time.

The target nucleic acid can be a nucleic acid present in an animal (e.g., human), plant, fungal (e.g., yeast), protozoan, bacterial, or viral species. For example, the target nucleic acid can be present in the genome of an organism of interest (e.g., on a chromosome) or on an extrachromosomal nucleic acid. In some embodiments, the target nucleic acid is an RNA, e.g., an mRNA. In particular embodiments, the target nucleic acid is specific for the organism of interest, i.e., the target nucleic acid is not found in other organisms or not found in organisms similar to the organism of interest.

The target nucleic acid can be present in a bacteria, e.g., a Gram-positive or a Gram-negative bacteria. Exemplary bacterial species include Acinetobacter sp. strain ATCC 5459, Acinetobacter calcoaceticus, Aerococcus viridans, Bacteroides fragilis, Bordetella pertussis, Bordetella parapertussis, Campylobacter jejuni, Clostridium difficile, Clostridium perfringens, Corynebacterium sp., Chlamydia pneumoniae, Chlamydia trachomatis, Citrobacter freundii, Enterobacter aerogenes, Enterococcus gallinarum, Enterococcus faecium, Enterobacter faecalis (e.g., ATCC 29212), Escherichia coli (e.g., ATCC 25927), Gardnerella vaginalis, Helicobacter pylori, Haemophilus influenzae (e.g., ATCC 49247), Klebsiella pneumoniae, Legionella pneumophila (e.g., ATCC 33495), Listeria monocytogenes (e.g., ATCC 7648), Micrococcus sp. strain ATCC 14396, Moraxella catarrhalis, Mycobactcrium kansasii, Mycobactcrium gordonac, Mycobacterium fortuitum, Mycoplasma pneumoniae, Mycoplasma hominis, Neisseria meningitis (e.g., ATCC 6250), Neisseria gonorrhoeae, Oligella urethralis, Pasteurella multocida, Pseudomonas aeruginosa (e.g., ATCC 10145), Propionibacterium acnes, Proteus mirabilis, Proteus vulgaris, Salmonella sp. strain ATCC 31194, Salmonella typhimurium, Serratia marcescens (e.g., ATCC 8101), Staphylococcus aureus (e.g., ATCC 25923), Staphylococcus epidermidis (e.g., ATCC 12228), Staphylococcus lugdunensis, Staphylococcus saprophyticus, Streptococcus pneumoniae (e.g., ATCC 49619), Streptococcus pyogenes, Streptococcus agalactiae (e.g., ATCC 13813), Treponema palliduma, Viridans group streptococci (e.g., ATCC 10556), Bacillus anthracis, Bacillus cereus, Francisella philomiragia (GA01-2810), Francisella tularensis (LVSB), Yersinia pseudotuberculosis (PB1/+), Yersinia enterocolitica, O:9 serotype, orYersinia pestis (P14−). In some embodiments, the target nucleic acid is present in a species of a bacterial genus selected from Acinetobacter, Aerococcus, Bacteroides, Bordetella, Campylobacter, Clostridium, Corynebacterium, Chlamydia, Citrobacter, Enterobacter, Enterococcus, Escherichia, Helicobacter, Haemophilus, Klebsiella, Legionella, Listeria, Micrococcus, Mobilincus, Moraxella, Mycobacterium, Mycoplasma, Neisseria, Oligella, Pasteurella, Prevotella, Porphyromonas, Pseudomonas, Propionibacterium, Proteus, Salmonella, Serratia, Staphylococcus, Streptococcus, Treponema, Bacillus, Francisella, or Yersinia. In some embodiments, the target nucleic acid is found in Group A Streptococcus or Group B Streptococcus.

Exemplary chlamydial target nucleic acids include sequences found on chlamydial cryptic plasmids.

Exemplary M. tuberculosis target nucleic acids include sequences found in IS6110 (see US 5,731,150) and/or IS1081 (see Bahador et al., 2005, Res. J. Agr. Biol. Sci., 1:142-145).

Exemplary N. gonorrhea target nucleic acids include sequences found in NGO0469 (see Piekarowicz et al., 2007, BMC Microbiol., 7:66) and NGO0470.

Exemplary Group A Streptococcus target nucleic acids include sequences found in Spyl258 (see Liu et al., 2005, Res. Microbiol., 156:564-567), Spy0193, lytA, psaA, and ply (see US 2010/0234245).

Exemplary Group B Streptococcus target nucleic acids include sequences found in the cfb gene (see Podbielski et al., 1994, Med. Microbiol. Immunol., 183:239-256).

In some embodiments, the target nucleic acid is a viral nucleic acid. For example, the viral nucleic acid can be found in human immunodeficiency virus (HIV), influenza virus, or dengue virus. Exemplary HIV target nucleic acids include sequences found in the Pol region.

In some embodiments, the target nucleic acid is a protozoan nucleic acid. For example, the protozoan nucleic acid can be found inPlasmodium spp., Leishmania spp., Trypanosoma brucei gambiense, Trypanosoma brucei rhodesiense, Trypanosoma cruzi, Entamoeba spp., Toxoplasma spp., Trichomonas vaginalis, and Giardia duodenalis.

In some embodiments, the target nucleic acid is a mammalian (e.g., human) nucleic acid. For example, the mammalian nucleic acid can be found in circulating tumor cells, epithelial cells, or fibroblasts.

In some embodiments, the target nucleic acid is a fungal (e.g., yeast) nucleic acid. For example, the fungal nucleic acid can be found in Candida spp. (e.g., Candida albicans).

Detecting the amplified product typically includes the use of labeled probes that are sufficiently complementary and hybridize to the amplified product corresponding to the target nucleic acid. Thus, the presence, amount, and/or identity of the amplified product can be detected by hybridizing a labeled probe, such as a fluorescently labeled probe, complementary to the amplified product. In some embodiments, the detection of a target nucleic acid sequence of interest, includes the combined use of an isothermal amplification method and a labeled probe such that the product is measured in real time. In another embodiment, the detection of an amplified target nucleic acid sequence of interest includes the transfer of the amplified target nucleic acid to a solid support, such as a membrane, and probing the membrane with a probe, for example a labeled probe, that is complementary to the amplified target nucleic acid sequence. In yet another embodiment, the detection of an amplified target nucleic acid sequence of interest includes the hybridization of a labeled amplified target nucleic acid to probes that are arrayed in a predetermined array with an addressable location and that are complementary to the amplified target nucleic acid.

Typically, one or more primers are utilized in an amplification reaction. Amplification of a target nucleic acid involves contacting the target nucleic acid with one or more primers that are capable of hybridizing to and directing the amplification of the target nucleic acid. In some embodiments, the sample is contacted with a pair of primers that include a forward and reverse primer that both hybridize to the target nucleic.

Real-time amplification monitors the fluorescence emitted during the reaction as an indicator of amplicon production as opposed to the endpoint detection. The real-time progress of the reaction can be viewed in some systems. Typically, real-time methods involve the detection of a fluorescent reporter. Typically, the fluorescent reporter's signal increases in direct proportion to the amount of amplification product in a reaction. By recording the amount of fluorescence emission at each cycle, it is possible to monitor the amplification reaction during exponential phase where the first significant increase in the amount of amplified product correlates to the initial amount of target template. The higher the starting copy number of the nucleic acid target, the sooner a significant increase in fluorescence is observed.

In some embodiments, the fluorescently-labeled probes rely upon fluorescence resonance energy transfer (FRET), or in a change in the fluorescence emission wavelength of a sample, as a method to detect hybridization of a DNA probe to the amplified target nucleic acid in real-time. For example, FRET that occurs between fluorogenic labels on different probes (for example, using HybProbes) or between a fluorophore and a non-fluorescent quencher on the same probe (for example, using a molecular beacon or a TAQMAN® probe) can identify a probe that specifically hybridizes to the DNA sequence of interest and in this way can detect the presence, and/or amount of the target nucleic acid in a sample. In some embodiments, the fluorescently-labeled DNA probes used to identify amplification products have spectrally distinct emission wavelengths, thus allowing them to be distinguished within the same reaction tube, for example in multiplex reactions. For example, multiplex reactions permit the simultaneous detection of the amplification products of two or more target nucleic acids even another nucleic acid, such as a control nucleic acid.

In some embodiments, a probe specific for the target nucleic acid is detectably labeled, either with an isotopic or non-isotopic label; in alternative embodiments, the amplified target nucleic acid is labeled. The probe can be detected as an indicator of the target nucleic acid species, e.g., an amplified product of the target nucleic acid species. Non-isotopic labels can, for instance, comprise a fluorescent or luminescent molecule, or an enzyme, co-factor, enzyme substrate, or hapten. The probe can be incubated with a single-stranded or double-stranded preparation of RNA, DNA, or a mixture of both, and hybridization determined. In some examples, the hybridization results in a detectable change in signal such as in increase or decrease in signal, for example from the labeled probe. Thus, detecting hybridization comprises detecting a change in signal from the labeled probe during or after hybridization relative to signal from the label before hybridization.

In some methods, the amplified product may be detected using a flow strip. In some embodiments, one detectable label produces a color and the second label is an epitope which is recognized by an immobilized antibody. A product containing both labels will attach to an immobilized antibody and produce a color at the location of the immobilized antibody. An assay based on this detection method may be, for example, a flow strip (dip stick) which can be applied to the whole isothermal amplification reaction. A positive amplification will produce a band on the flow strip as an indicator of amplification of the target nucleic acid species, while a negative amplification would not produce any color band.

In some embodiments, the amount (e.g., number of copies) of a target nucleic acid can be approximately quantified using the methods disclosed herein. For example, a known quantity of the target nucleic acid can be amplified in a parallel reaction and the amount of amplified product obtained from the sample can be compared to the amount of amplified product obtained in the parallel reaction. In some embodiments, several known quantities of the target nucleic acid can be amplified in multiple parallel reactions and the amount of amplified product obtained form the sample can be compared to the amount of amplified product obtained in the parallel reactions. Assuming that the target nucleic acid in the sample is similarly available to the reaction components as the target nucleic acid in the parallel reactions, the amount of target nucleic acid in the sample can be approximately quantified using these methods.

The reaction components for the methods disclosed herein can be supplied in the form of a kit for use in the detection of target nucleic acids. In such a kit, an appropriate amount of one or more reaction components is provided in one or more containers or held on a substrate. A nucleic acid probe and/or primer specific for a target nucleic acid may also be provided. The reaction components, nucleic acid probe, and/or primer can be suspended in an aqueous solution or as a freeze-dried or lyophilized powder, pellet, or bead, for instance. The container(s) in which the components, etc. are supplied can be any conventional container that is capable of holding the supplied form, for instance, microfuge tubes, ampoules, or bottles or integral testing devices such microfluidic devices, lateral flow, or other similar devices. The kits can include either labeled or unlabeled nucleic acid probes for use in detection of target nucleic acids. In some embodiments, the kits can further include instructions to use the components in a method described herein, e.g., a method using a crude matrix without nucleic acid extraction and/or purification.

In some applications, one or more reaction components may be provided in pre-measured single use amounts in individual, typically disposable, tubes or equivalent containers. With such an arrangement, the sample to be tested for the presence of a target nucleic acid can be added to the individual tubes and amplification carried out directly.

The amount of a component supplied in the kit can be any appropriate amount, and may depend on the target market to which the product is directed. General guidelines for determining appropriate amounts may be found in Innis et al., Sambrook et al., and Ausubel et al.

### EXAMPLES

### Example 1

### Detection of Bacteria in a Crude Matrix

The ability to amplify nucleic acids in a crude sample was investigated. Salmonella typhimurium was grown in LB broth. Mid-exponential phase cultures were diluted to 100, 1000, or 10,000 cfu in The diluted cultures were lysed by mixing the samples with 2.5 μl 0.2 NaOH, 0.1% Triton X-100 for five minutes, followed by neutralization with 1 μl 1 M acetic acid. Control cultures (no lysis) were mixed with resuspension buffer for amplification. Two hundred copies of an invA PCR product were used as a positive control, and LB medium was used as a negative control. To each sample was added 3.5 μl each of 6 μM solutions of forward and reverse amplification primers (INVAF2, ccgtggtccagtttatcgttattaccaaaggt, SEQ ID NO:1 and INVAR2, ccctttccagtacgcttcgccgttcgcgcgcg, SEQ ID NO:2), 8.5 μl 20% PEG 35K, 2.5 magnesium acetate (280 mM), a lyophilized reaction pellet containing 1.25 μg creatine kinase, 23 μg UvsX, 5 μg UvsY, 24.25 μg Gp32, 6.65 μg ExoIII, 14.65 μg Poll, PEG 35000 (final concentration 5.5% w/v), Tris pH8.3 (final concentration 50 mM), DTT (final concentration 5 mM), phosphocreatine (final concentration 50 mM), ATP (final concentration 2.5 mM), trehalose (final concentration 5.7% w/v), and dNTPs (each final concentration 300 mM), detection probe attttactggatggtatgcceggtaaacagaQgHgFattgatgccgatt (Q =BHQ-1-dT; H=THF; F=Fluorescein-dT; 3′=biotin-TEG (15 atom triethylene glycol spacer); SEQ ID NO:3) and water to 50 μl total reaction volume. In the lysed samples, S. typhimurium was detected in all samples depending on the number of cells (FIG. 1B). The signal strength with 1000 cfu was much stronger than the control target DNA used at 200 copies, while the 100 cfu sample was slightly weaker than the control. This data suggests very much that most, if not all, the bacteria were lysed by the process and that their DNA was fully available to act as template in the amplification reaction. In the absence of a lysis step (FIG. 1A), amplification of the target was detected in one case when 10,000 cfu were used (possibly due to occasional genomic DNA contamination from rare lysis) but not otherwise. This example demonstrates that bacteria can be detected directly following straightforward alkaline lysis at high sensitivity from growth medium.

### Example 2

### Detection of Bacteria in Saliva Following Simple Lysis

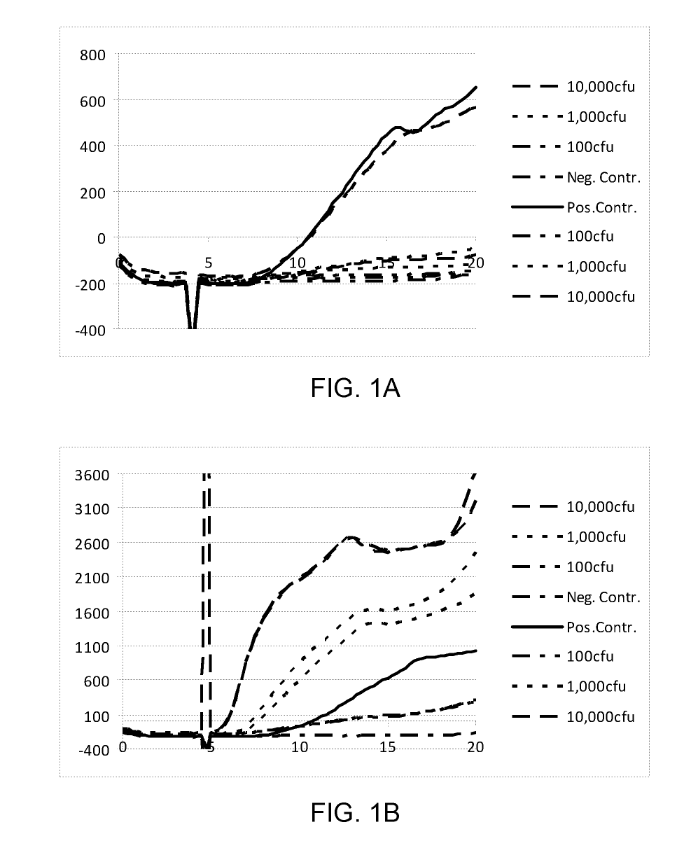

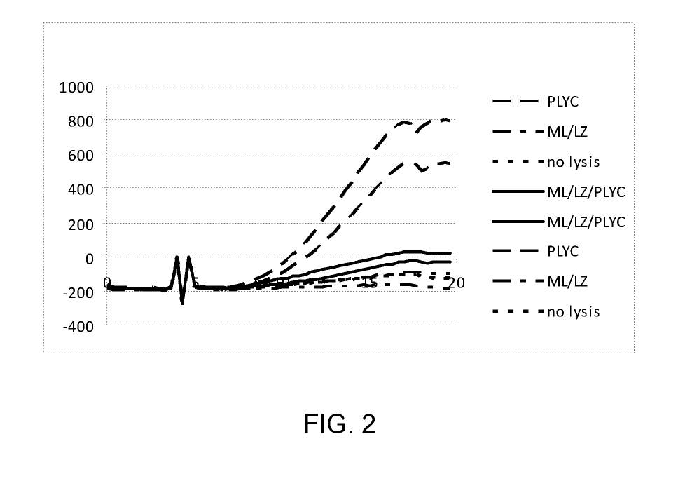

This example demonstrates another target and sample that can be detected without a requirement for nucleic acid extraction. In this experiment primers and probes developed for the detection of a Streptococcus A gene (Primers: PTSF31, CAAAACGTGTTAAAGATGGTGATGTGATTGCCG, SEQ ID NO:4; PTSR25, AAGGAGAGACCACTCTGCTTTTTGTTTGGCATA, SEQ ID NO:5; Probe: PTSP3, CAAAACGTGTTAAAGATGGTGATGTGATTGCCGTQAHFGGTATCACTGGTGAA G, Q=dT-BHQ2, H=THF, F=dT-TAMRA, 3′=C3-SPACER, SEQ ID NO:6) were used to investigate the ability to detect Strep A directly from saliva samples. Saliva was pooled from a number of individuals known to carry Strep A and used at a target copy number of 1000 cfu/ml of saliva. Twenty microliters of saliva (1000 cfu/ml) were mixed with 1 μl 0.1% Triton X-100 and a) water, b) 1 μl mutanolysin (50 U/μl) and 0.5 μl lysozyme (100 mg/ml), c) 2 μl PlyC (2.2 mg/ml) (Nelson et al., 2006, Proc. Natl. Acad. Sci. USA, 103:10765-70), or d) mutanolysin, lysozyme, and PlyC (amounts as in b and c). The reactions were prepared as in Example 1, except in a volume of 100 μl Strep A was able to be detected directly in saliva when the sample was incubated with the PlyC enzyme known to have a lytic effect on Strep A (FIG. 2). This was the case even when one fifth (20 microliters in 100 microliter final reaction volume) of the reaction was composed of saliva, and in this case can only contain about 50 micro-organisms within the reaction. This example demonstrates that even in a crude matrix comprising 20% saliva and without nucleic acid purification, RPA can provide remarkable sensitivity and robust kinetics.

### Example 3

### Detection of Bacteria in Unlysed Samples

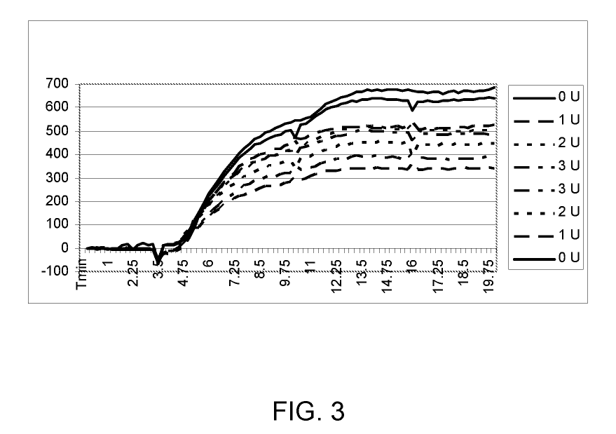

Staphylococcus aureus (S. aureus) was detected using primers and probes developed to detect the S. aureus nuc gene. A flocked swab (Copan #503CS01) was used to take a sample from the anterior nares of a known Staphylococcus aureus carrier. The swab was dunked into 500 μl resuspension buffer and then discarded. 46.5 μl aliquots of this swab liquid were added to 1 μl of 0, 1, 2, and 3 Units of lysostaphin. The 47.5 μl of swab liquid/lysostaphin were then used to resuspend freeze-dried ‘nue RPA reactions as described in Example 1 and also containing primers nucF10 (CTTTAGTTGTAGTTTCAAGTCTAAGTAGCTCAGCA, SEQ ID NO:7) and nucR6 (CATTAATTTAACCGTATCACCATCAATCGCTTTAA, SEQ ID NO:8) and the probe nucProbel (agtttcaagtctaagtagctcagcaaaRgHaQcacaaacagataa, wherein R=Tamra dT, H=THF or D-spacer (abasic site mimic), Q=BlackHoleQuencher2 dT, 3’=Biotin-TEG, SEQ ID NO:9). 2.5 μl 280 mM MgAc was added simultaneously to each reaction to start them. Reactions were run at 38° C. for 20 minutes with the samples being agitated by vortexing after 4 minutes. Surprisingly, the strongest signals were observed when no lysostaphin at all was added to the samples (FIG. 3). Addition of lysostaphin may have led to a small reduction in total signal intensity. This example demonstrates that lysis may not be necessary for amplification in some situations.

### Example 4

### Heat Treatment is Not Necessary for Amplification Reactions

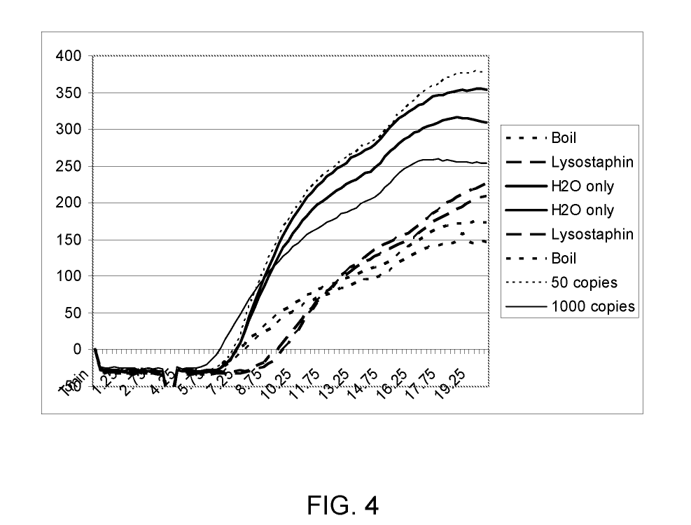

A flocked swab (Copan #516CS01) was used to take a sample from the anterior nares of a known S. aureus carrier. The swab was dunked into 350 μl water and then discarded. The swab liquid was then mixed and aliquotted into three lots of 99 Two aliquots had 1.65 μl water added and the third had 1.65 μl lysostaphin (43 Units/μl) added. The aliquots with water added were either boiled for 45 minutes or left at room temperature for 45 minutes. The lysostaphin aliquot was heated to 37° C. for 40 minutes and then boiled for 5 minutes to destroy any nucleases. 91.5 μl of each aliquot was added to 27 μl 20% PEG, 9 μl nucForwardPrimerl 0 (SEQ ID NO:7), 9 μl nucReversePrimer6 (SEQ ID NO:8) and 3 μl nuc probel (SEQ ID NO:9) to create reaction mixes. In duplicate, 46.5 μl each reaction mix was then used to resuspend freeze-dried Primer Free RPA reactions as described in Example 1. 2.5 μl 280 mM MgAc was added simultaneously to each reaction to start them. Reactions were run at 38° C. for 20 minutes with the samples being agitated by vortexing after 4 minutes. Two positive control reactions using the same primers and probes and known copy numbers of nuc PCR product were also run. Interestingly, in this case the strongest signals were found the sample which was not subjected to either boiling or to lysostaphin treatment followed by boiling (FIG. 4). The act of boiling in this case actually led to a decrease in overall sensitivity, perhaps either due to damage to DNA or to release of some inhibitory components. Furthermore, incubation for some period of time with lysostaphin before short boiling gave a further reduction in sensitivity. In the case of boiling alone the time of onset was similar to the unlysed sample arguing that the accessible copy number was the same, but that perhaps some inhibitor was released that quashed the strength of the final fluorescent signal. In the case of the lysostaphin pre-treatment the signal was also later, suggesting that the accessible target copy number had decreased, possibly due to DNA degradation during the incubation. Taken collectively, these data argue that most or all potential target DNA is available to the RPA reagents when sample is placed into the RPA reaction and that if anything pre-lysis by heating or enzymes only lowers the available copy number or releases undesirable inhibitors. This example further demonstrates that RPA can be a suitable technique for the direct detection of S. aureus in biological samples compared to other techniques requiring initial denaturation.

### Example 5

### DNA Purification is Not Necessary for Amplification Reactions

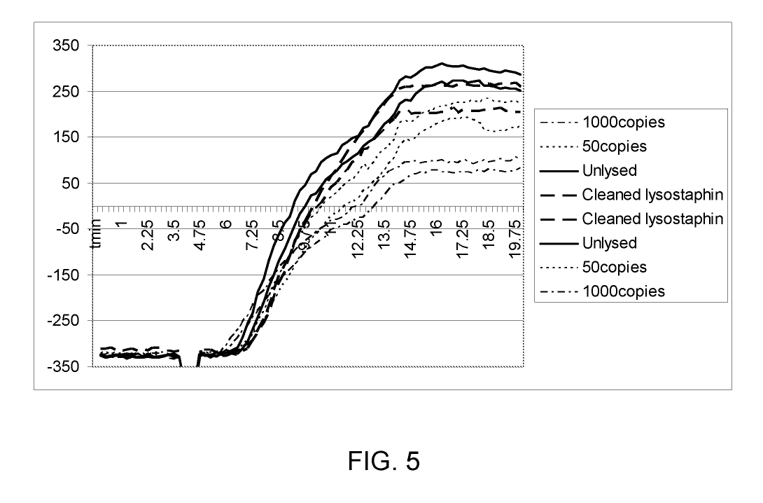

A flocked swab (Copan #516CS01) was used to take a sample from the anterior nares of a known S. aureus carrier. The swab was dunked into 300 μl water and then discarded. The swab liquid was then mixed and aliquotted into two lots of 100 μl. The first aliquot had 2 μl lysostaphin (43 Units/μl) added, the second lot was left alone. The lysostaphin aliquot was heated to 37° C. for 45 minutes and then boiled for 5 minutes to destroy any nucleases. 3 μg of human genomic DNA (carrier DNA) was added to the lysed swab liquid and then all of the DNA extracted using QlAgen's Dneasy Mini protocol and eluted into 100 μl water. 30.5 μl of the unlysed and lysed aliquots were added to 9 μl 20% PEG, 3 μl nucForwardPrimer10 (SEQ ID NO:7), 3 μl nucReversePrimer6 (SEQ ID NO:8) and 1 μl nuc probe1 (SEQ ID NO:9) to create reaction mixes. 46.5 μl of each reaction mix was then used to resuspend freeze-dried Primer Free RPA reactions as described in Example 1. 2.5 μl 280 mM MgAc was added simultaneously to each reaction to start them. The reactions were run at 38° C. for 20 minutes with the samples being agitated by vortexing after 4 minutes. Duplicate positive control reactions using the same primers and probes and known copy numbers of nuc PCR product were also run. The purified and eluted DNA performed similarly to the unlysed/untreated sample (albeit with a slightly later onset indicating a lower copy number) (FIG. 5). As the cleanup step eliminated the poor amplification curve noted with boiling alone it suggests that boiling may release an inhibitor from S. aureus which can subsequently be removed by a clean-up protocol. However, as noted in the earlier experiment, this damaging reagent is simply not encountered if the sample is used directly in RPA reactions while the target DNA seems to be fully accessible as the copy number likely falls when processing occurs as indicated by the later onset following DNA extraction.

### Example 6

### Detection of Nucleic Acids in Unlysed Cells

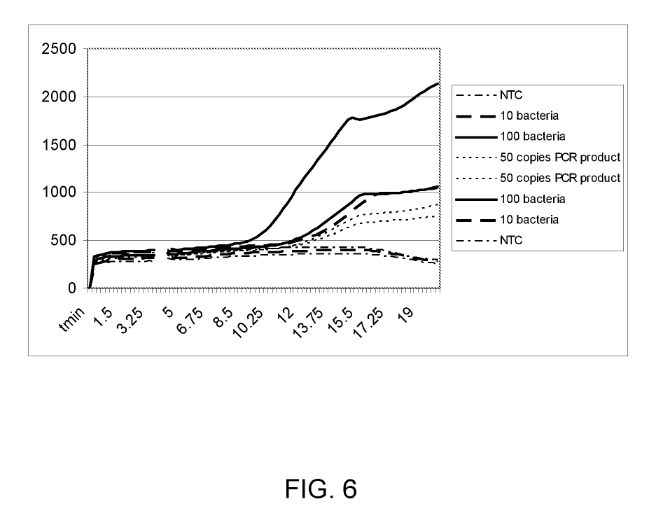

Inactivated methicillin resistant Staphylococcus aurcus (MRSA) from the Quality Control for Molecular Diagnostics panel was diluted and added in known quantities directly to RPA reactions. 27.5 μl of water, 1 μl of DNA/bacteria/H₂O, 9 μl 20% PEG, 1.6 μl orfX_ForwardPrimer10+6 (CGTCTTACAACGCAGTAACTACGCACTATCATTCA, SEQ ID NO:10), 1.6 μl orfX_ForwardPrimerl (CAAAATGACATTCCCACATCAAATGATGCGGGTTG, SEQ ID NO:11), 1.6 μl mrej-i_ReversePrimer4 (CTGCGGAGGCTAACTATGTCAAAAATCATGAACCT, SEQ ID NO:12), 1.6 μl mrej-ii_ReversePrimer4-1 (ACATTCAAAATCCCTTTATGAAGCGGCTGAAAAAA, SEQ ID NO:13), 1.6 μl mrej-iii_ReversePrimer5 (ATGTAATTCCTCCACATCTCATTAAATTTTTAAAT, SEQ ID NO:14) and 1 μl SAFAMprobe3 (5′-TGACATTCCCACATCAAATGATGCGGGTbGxGfTAATTGARCAAGT-3′, where f=Fam dT, x=THF or D-spacer (abasic site mimic), b=BHQ1 dT, and 3′=Biotin-TEG, SEQ ID NO:15) (all at 1.6 μM) were used to resuspend freeze-dried Primer Free RPA reactions as described in Example 1. 2.5 μl 280 mIVI MgAc was added simultaneously to each reaction to start them. Reactions were run at 38° C. for 20 minutes with the samples being agitated by vortexing after 4 minutes. The target nucleic acid was routinely detected when 100 bacterial targets were included and sporadically when 10 bacterial targets were included (FIG. 6). These data are in agreement with the notion that most or all of the potential DNA targets in the sample were available—indeed the signals from the 100 targets initiated earlier than from the 50 copy template control, and the 10 copies initiated slightly later, and therefore it is likely that all the targets were available. The failure of one 10 target sample may be due to bacterial clumping affecting the presence or absence of any targets in the absence of extraction, or due to the overall cut-off sensitivity of this RPA test for nuc being at around 10 copies.

### Example 7

### Detection of Mycoplasma Nucleic Acids Without Lysis

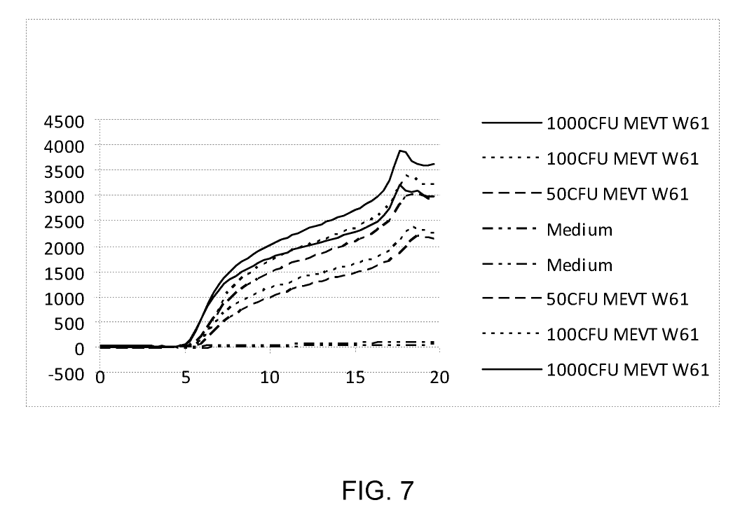

FIG. 7 shows direct detection of another bacterial target in the absence of any initial lysis treatment. In this case primers and probes developed to detect porcine mycoplasma (Forward primer: Mhy183F36 GCAAAAGATAGTTCAACTAATCAATATGTAAGT (SEQ ID NO:16), Reverse primer: Mhy183R124 ACTTCATCTGGGCTAGCTAAAATTTCACGGGCA (SEQ ID NO:17), 5′-TCATCTGGGCTAGCTAAAATTTCAC GGGCACTTQGHCFAAGATCTGCTTTTA-3′, F=TAMRA dT, H=THF (abasic site mimic), Q=BHQ-2 dT (SEQ ID NO:18) were used to assess their ability to detect mycoplasma. Heat-inactivated mycoplasma MEVT W61 was obtained from Mycoplasma Experience UK, present (titred) on agarose. Flocked swabs were used to take a sample which was dunked directly into RPA rehydration buffer. The buffer was diluted to 1000, 100 and 50 cfu mycoplasma and used to rehydrate RPA reactions as described in Example 1 configured to amplify the specific mycoplasma target. Included in this experiment is an internal control measured in another fluorescent channel which targets an artificial plasmid sequence placed into the reaction environment. In all cases, and even down to a sensitivity of 50 cfu, the test was able to detect the porcine mycoplasma sequences efficiently (FIG. 7).

### Example 8

### Detection of M. tuberculosis

To test for the presence of M. tuberculosis in a patient, a sputum sample is obtained from the patient and mixed with resuspension buffer. The mixture is used as is or subjected to lysis. The mixture is subjected to RPA reaction to amplify nucleic acid species corresponding to IS6110 (see U.S. Pat. No. 5,731,150) and/or IS1081 (see Bahador et al., 2005, Res. J. Agr. Biol. Sci., 1:142-145). Detection of an amplification product corresponding to IS6110 or IS1081 indicates the presence of M. tuberculosis in the patient sample.

### Example 9

### Detection of Group A Streptococcus

To test for the presence of Group A Streptococcus in a patient, a throat swab or saliva sample is obtained from the patient and mixed with resuspension buffer. The mixture is used as is or subjected to lysis. The mixture is subjected to RPA reaction to amplify nucleic acid species corresponding to Spyl258 (see Liu et al., 2005, Res. Microbiol., 156:564-567) and/or Spy0193. Detection of an amplification product corresponding to Spyl258 or Spy0193 indicates the presence of Group AStreptococcus in the patient sample.

### Example 10

### Detection of N. gonorrhea

To test for the presence of N. gonorrhea in a patient, a vaginal swab or urine sample is obtained from the patient and mixed with resuspension buffer. The mixture is used as is or subjected to lysis. The mixture is subjected to RPA reaction to amplify nucleic acid species corresponding to NGO0469 (see Piekarowicz et al., 2007, BMC Microbiol., 7:66) and/or NGO0470. Detection of an amplification product corresponding to NGO0469 or NGO0470 indicates the presence of N. gonorrhea in the patient sample.

### Example 11

### Detection of Chlamydia

To test for the presence of chlamydia in a patient, a vaginal swab or urine sample is obtained from the patient and mixed with resuspension buffer. The mixture is used as is or subjected to lysis. The mixture is subjected to RPA reaction to amplify nucleic acid species corresponding to the chlamydia cryptic plasmid (see Hatt et al., 1988, Nucleic Acids Res. 16:4053-67). Detection of an amplification product corresponding to the cryptic plasmid indicates the presence of chlamydia in the patient sample.

### Example 12

### Detection of Group B Streptococcus

To test for the presence of Group B Streptococcus in a patient, a vaginal or rectal swab is obtained from the patient and mixed with resuspension buffer. The mixture is used as is or subjected to lysis. The mixture is subjected to RPA reaction to amplify nucleic acid species corresponding to the cfb gene (see Podbielski et al., 1994, Med. Microbiol. Immunol., 183:239-256). Detection of an amplification product corresponding to the cfb gene indicates the presence of Group B Streptococcus in the patient sample.

### Example 13

### Detection of HIV

To test for the presence of HIV in a patient, a blood sample (e.g., whole blood or buffy coat) is obtained from the patient and mixed with resuspension buffer. The mixture is used as is or subjected to lysis. The mixture is subjected to RPA reaction to amplify nucleic acid species corresponding to the Pol region. Detection of an amplification product corresponding to the Pol region indicates the presence of HIV in the patient sample.

### OTHER EMBODIMENTS

A number of embodiments of the invention have been described. Nevertheless, it will be understood that various modifications may be made without departing from the spirit and scope of the invention. Accordingly, other embodiments are within the scope of the following claims.

1-54. (canceled)

55. A method for determining the presence or absence of a pathogen in a stool sample without nucleic acid extraction or purification, comprising;
i) applying a stool sample to an assay device;
ii) lysing the sample within the assay device to release intracellular nucleic acid of the pathogen;
iii) contacting the sample with a recombinase polymerase amplification (RPA) reagent mixture within the assay device, wherein the RPA reagent mixture comprises primers specific for the pathogen nucleic acid, a UvsX recombinase, and a UvsY loading agent;
iv) amplifying the target nucleic acid; and
v) determining the presence or absence of the pathogen nucleic acid based on an increased signal from a nucleic acid probe within the assay device.

56. The method of claim 55, wherein the assay device is selected from the group consisting of a microfluidic device, a lateral flow device, and similar such assay device.

57. The method of claim 55, wherein the pathogen is selected from the group consisting of the species Acinetobacter, Aerococcus, Bacteroides, Bordetella, Campylobacter, Clostridium, Corynebacterium, Chlamydia, Citrobacter, Enterobacter, Enterococcus, Escherichia, Helicobacter, Haemophilus, Klebsiella, Legionella, Listeria, Micrococcus, Mobilincus, Moraxella, Mycobacterium, Mycoplasma, Neisseria, Oligella, Pasteurella, Prevotella, Porphyromonas, Pseudomonas, Propionibacterium, Proteus, Salmonella, Serratia, Staphylococcus, Streptococcus, Treponema, Bacillus, Francisella, and Yersinia.

58. The method of claim 55, wherein lysing the sample comprises contacting the sample with a detergent and/or a lytic enzyme.

59. The method of claim 58, wherein the detergent comprises Triton-X 100.

60. The method of claim 58, wherein the lytic enzyme comprises bacteriophage lysin.

61. The method of claim 60, wherein the bacteriophage lysin comprises Streptococcal Ci bacteriophage lysin (PlyC).

62. The method of claim 55, wherein the RPA reagent mixture further comprises a gp32 single strand binding protein, a polymerase, and a crowding agent.

63. The method of claim 55, wherein the UvsX is a T4 phage UvsX and the UvsY is a T4 phage UvsY.

64. The method of claim 62, wherein the gp32 is a T4 phage gp32, the polymerase is a Bacillus subtilis polymerase I large fragment, and the crowding agent is polyethylene glycol (PEG).

65. The method of claim 55, wherein the RPA reagent mixture is provided in dried form within the assay device.

66. The method of claim 65, wherein the dried RPA reagent mixture is provided as freeze-dried or lyophilized powder, pellet or bead.

67. The method of claim 55, wherein the nucleic acid probe is a fluorescent probe.

68. The method of claim 67, wherein the fluorescent probe is selected from the group consisting of fluorescein, TAMRA, and Fam.
